(19) United States
(12) Patent Application Publication
(10) Pub. No.: US 2008/0221411 A1
(43) Pub. Date: Sep. 11, 2008

(54) System and method for tissue hydration estimation

(75) Inventors: Gilbert Hausmann, Felton, CA (US); Shannon E. Campbell, Oakland, CA (US); Allison Ferro, Fremont, CA (US)

Correspondence Address: NELLCOR PURITAN BENNETT LLC;ATTN: IP LEGAL, 60 Middletown Avenue, North Haven, CT, 06473, US

(73) Assignee: Nellcor Puritan Bennett LLC

(21) Appl. No.: 11/716,443

(22) Filed: Mar. 9, 2007

(51) Int. Cl. A61B 5/00

(52) U.S. Cl. 600/310

(57) ABSTRACT

A system and method are provided for determining tissue hydration. The method includes transmitting electromagnetic radiation at tissue and detecting the absorption spectrum of the tissue using a spectrum analyzer located in a sensor. Further, the method includes providing a signal correlative to the absorption spectrum from the spectrum analyzer to a monitor and processing the signal to determine an amount of water content in the tissue.

### TECHNICAL FIELD

The present invention relates generally to determining physiological parameters and, more particularly, to determining tissue hydration.

### BACKGROUND

This section is intended to introduce the reader to various aspects of art that may be related to various aspects of the present invention, which are described and/or claimed below. This discussion is believed to be helpful in providing the reader with background information to facilitate a better understanding of the various aspects of the present invention. Accordingly, it should be understood that these statements are to be read in this light, and not as admissions of prior art.

In the field of medicine, doctors and other health care professionals often desire to know certain analyte levels and physiological characteristics of their patients. For example, doctors may want to know the level of a patient's hydration, hematocrit, skin cholesterol, bilirubin, and carbon dioxide, as well as injected anesthetic agents, among others. Once the analyte levels and/or physiological characteristics are known, the doctors and other health care professionals are able to properly assess an individual's condition and provide the best possible health care. Accordingly, a wide variety of devices and techniques have been developed for determining and monitoring analyte levels and physiological characteristics. Such monitoring devices have become an indispensable part of modern medicine.

While some techniques for the assessment of analytes require invasive procedures such as extraction of fluids using a syringe and needles, non-invasive devices and techniques provide increased comfort to the patient and ease of use for the doctors or health care professionals. Some non-invasive devices implement spectroscopic techniques. However, spectrophotometers used to implement the spectroscopic techniques are generally large, expensive, and delicate.

### SUMMARY

Certain aspects commensurate in scope with the originally claimed invention are set forth below. It should be understood that these aspects are presented merely to provide the reader with a brief summary of certain forms the invention might take and that these aspects are not intended to limit the scope of the invention. Indeed, the invention may encompass a variety of aspects that may not be set forth below.

In accordance with one aspect of the present invention, there is provided a method for determining tissue hydration. The method includes transmitting electromagnetic radiation at tissue and detecting the absorption spectrum of the tissue using a spectrometer located in a sensor. The absorption spectrum is provided to a monitor and interpreted to determine an amount of water content in the tissue.

### BRIEF DESCRIPTION OF THE DRAWINGS

Certain exemplary embodiments are described in the following detailed description and in reference to the drawings in which:

FIG. 1 illustrates a system for measuring tissue hydration in accordance with an exemplary embodiment of the present invention;

FIG. 2 illustrates a block diagram of the system of FIG. 1 in accordance with an exemplary embodiment of the present invention;

FIG. 3 illustrates layers of a solid state micro spectrometer in accordance with an exemplary embodiment of the present invention;

FIG. 4 is an illustration of filters of the solid state spectrometer of FIG. 3 in accordance with an exemplary embodiment of the present invention.

FIG. 5 illustrates a spectrograph of water generated by the solid state micro spectrometer of FIG. 3;

FIG. 6 illustrates a cross-sectional view of a micro-electro-mechanical systems (MEMS) spectrum analyzer in accordance with an alternative exemplary embodiment of the present invention; and

FIG. 7 illustrates a spectrograph of water generated by the MEMS spectrum analyzer of FIG. 6.

### DETAILED DESCRIPTION

One or more specific embodiments of the present invention will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

In accordance with the present technique, a method and apparatus are provided for estimating analyte concentration using spectroscopic techniques. Specifically, analyte levels may be estimated using system implementing a solid state spectrometer or a micro-electromechanical system (MEMS) detector. Among others, the determined analyte levels may include water, hematocrit, skin cholesterol, bilirubin, and carbon dioxide, as well as injected anesthetic agents. In an exemplary embodiment, the method and apparatus implement a broadband source of electromagnetic radiation, such as a white light. In another exemplary embodiment, a plurality of narrow band emitters, such as light emitting diodes (LEDs), operating at unique wavelengths are implemented.

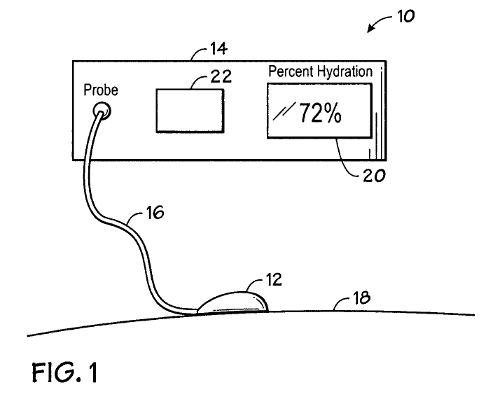

Referring to FIG. 1, a system configured to measure tissue hydration in accordance with an exemplary embodiment of the present invention is shown and generally designated by the reference numeral 10. The system 10 has a sensor 12 communicatively coupled with a monitor 14 via a cable 16. The sensor 12 is configured to be optically coupled with tissue 18 so that it may non-invasively probe the tissue 18 with electromagnetic radiation and generate a spectrum representative of the absorption and/or scattering of the electromagnetic radiation by the tissue 18. The absorbance spectrum is communicated via the cable 16 to the monitor 14 for processing, as described in greater detail below. In an alternative embodiment (not shown), the sensor 12 may be integrated with the monitor 14 in a single housing and configured to be carried by a caregiver, such as a nurse or a doctor for example. In yet another alternative embodiment, the sensor 12 and the monitor 14 may be configured to communicate wirelessly. The sensor 12 could then be transported by a caregiver independent of the monitor 14.

The monitor 14 may use the spectrum to calculate one or more physiological parameters and analyte levels including water, hematocrit, skin cholesterol, bilirubin, and carbon dioxide, as well as injected anesthetic agents, among others. The analyte levels may be indicative of the percentage of the analyte relative to other constituents in the probed tissue. With particular regard to water levels, a ratio of the water to other constituents present in the tissue may be determined and correlated with a hydration index. Specifically, for example, the monitor 14 may implement one of the methods for measuring water in tissue by NIR spectroscopy as described in U.S. Pat. No. 6,591,122; U.S. Pub. No. 2003-0220548; U.S. Pub. No. 2004-0230106; U.S. Pub. No. 2005-0203357; U.S. Ser. No. 60/857045; U.S. Ser. No. 11/283,506; and U.S. Ser. No. 11/282,947 all of which are incorporated herein by reference. Alternatively, the monitor 14 may implement techniques for measuring the analyte concentrations using a spectral bandwidth absorption, as described in U.S. Pub. Ser. No. 11/528,154, which is also incorporated herein by reference.

Referring again to FIG. 1, a display 20 is provided with the monitor 14 to indicate the physiological parameters, such as percent hydration, of the tissue 18 that was probed by the sensor 12. The system 10 may also be configured to receive input via a keyboard 22, for example, to allow a user to communicate with the system 10. For example, the keyboard 22, or other devices, can be used to enter baseline hydration values or threshold levels that may be indicative of a certain condition such as dehydration or over-hydration. Additionally, the keyboard 22 may be used to indicate to the system 10 what part of the body the sensor 12 will be probing, as the coefficients used in calculating the physiological parameters may be site specific.

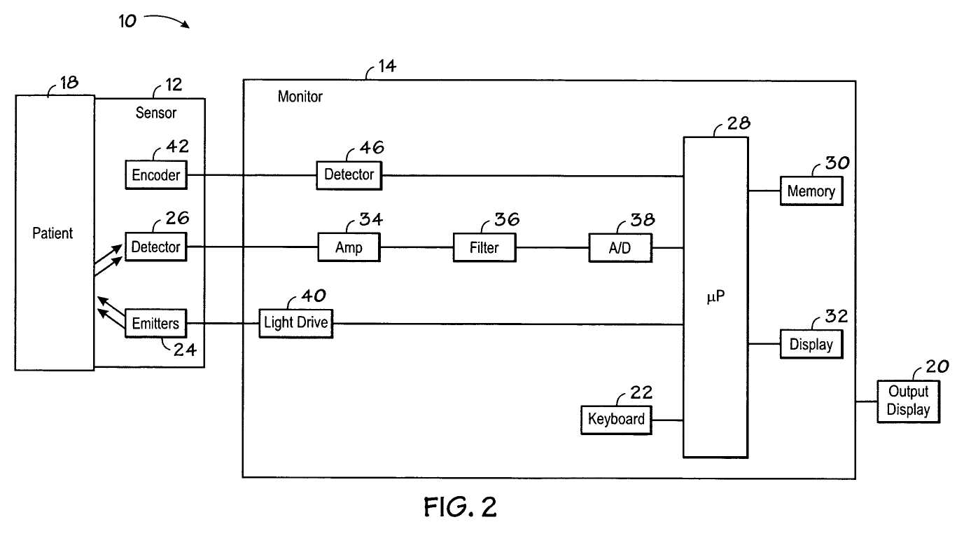

Turning to FIG. 2, a block diagram of the system 10 is illustrated in accordance with an exemplary embodiment of the present invention. As can be seen, the system 10 includes the sensor unit 12 having an emitter 24 configured to transmit electromagnetic radiation, such as light, into tissue 18 of a patient. The electromagnetic radiation is scattered and absorbed by the various constituents of the patient's tissues, such as water and protein. The sensor 12 also has a spectrum analyzer 26 configured to detect the scattered and reflected light and to generate a corresponding absorbance spectrum. The sensor 12 electrically communicates the absorbance spectrum from the spectrum analyzer 26 into the monitor 14, where the spectrum is processed.

Water has distinctive absorption bands in the near-infrared (NIR) spectrum, meaning it absorbs particular wavelengths of electromagnetic radiation in the NIR region of the electromagnetic spectrum. In order to differentiate water from other constituents that may be present in the tissue, a continuous or broadband light source, such as a white light source, for example, may be used. In an alternative embodiment, multiple discrete NIR wavelengths may be used operating near water spectral absorption bands. Specifically, in one exemplary embodiment four LEDs may be used to provide four different NIR wavelengths near the absorption bands of water to provide a nearly continuous spectrum near the water absorption bands to allow for differentiation of water from other tissue constituents. Additionally, other alternative light sources may be implemented, such as vertical-cavity surface-emitting lasers (VCSELs), for example.

The sensor 12 may be configured as a transmission type sensor or a reflectance type sensor. The sensor 12, shown in FIG. 1, is configured as a reflectance type sensor, as the emitter 22 and the spectrum analyzer 24 are in the same plane and the electromagnetic energy emitted from emitter 22 is reflected back to the spectrum analyzer 24 by the tissue 18. In an alternative exemplary embodiment, a transmission type sensor may be used. The transmission type sensor is configured so that the spectrum analyzer 24 is in a plane that is spaced from and substantially parallel with the plane in which the emitter 22 resides. During operation, a light path is created between the emitter 22 and spectrum analyzer 24 as electromagnetic energy is transmitted through the tissue. As with the reflection type sensor 12, the spectral power distribution of the detected electromagnetic energy can be used to determine the percent hydration of the tissue. In alternative embodiments, the emitter 22 and spectrum analyzer 24 may be positioned so that the electromagnetic energy enters the tissue at an angle. The angle may be known and any measurements may be adjusted to compensate for the angle.

The spectrum analyzer 24 may be a solid state spectrometer, such as those available from NanoLambda. The solid state spectrometer may have narrow-band micro-filters covering one or more cells. The narrow-band micro-filters allow only a certain wavelength of light through, thereby producing a curve representative of the light detected at that wavelength. The multiple micro-filters may have adjacent transmission bands allowing for an assessment of the light intensity of the spectral components of the analyzed light. Because of the filtering, however, the resulting spectrum of detected light may be choppy and discontinuous.

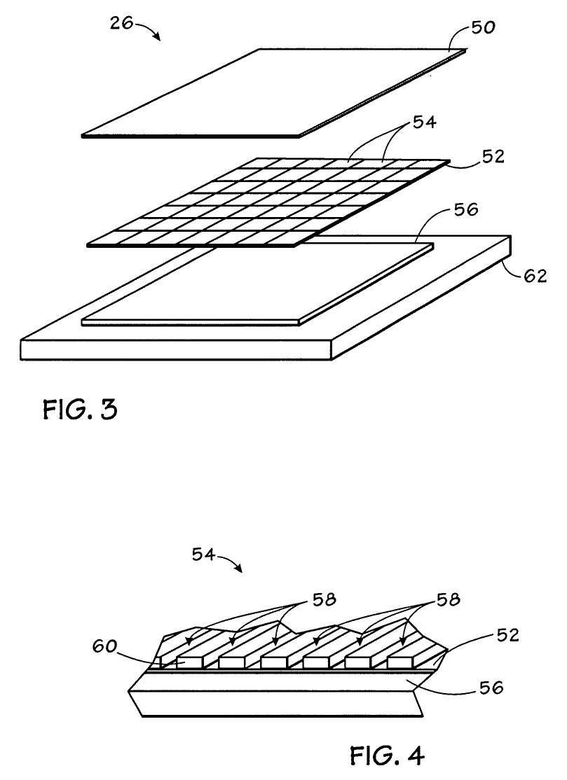

Turning to FIG. 3, various layers of the solid state spectrometer 26 are illustrated. The solid state spectrometer 26 has an optical window 50 as a first layer which serves a dual purpose. First, it allows electromagnetic radiation to enter into the solid state spectrometer 26. Second, it protects the functional parts of the spectrometer 26 from potential contaminants. Additionally, the optical window 50 may be polarized, so that light oriented differently from the polarized window is not allowed to pass into the spectrometer 26. The light allowed to pass into the spectrometer may, thus, have a known polarization and changes in the polarization due to traversing the tissue of interest may be determined and used in the assessment of the tissue.

The second layer is a metal nano wire array filter 52. The metal nano wire array filter 52 is an array of nano-sized metal filters 54 which filter the electromagnetic radiation that passes through the optical window 50. Each of the nano-sized filters may be configured to allow a particular wavelength of electromagnetic radiation or a narrow band of electromagnetic radiation to pass through to a detector array 56.

As shown in FIG. 4, the nano-sized filters 54 may include a number of nano-sized metal pieces 60 arranged to allow only a narrow bandwidth of electromagnetic radiation through apertures 58. The electromagnetic radiation that passes through the apertures 58 impinges upon the detector array 56 which may provide an indication of the amplitude of the electromagnetic radiation detected for that particular wavelength of narrow spectrum of electromagnetic radiation.

When fully assembled, the solid state spectrometer uses the filters 54 in conjunction with the detector array 56 to detect the electromagnetic radiation of the NIR spectrum for the determination of skin water content or hydration levels. All of the various layers of the solid state spectrometer 26 may be contained in single package 62 to provide protection and to allow the solid state spectrometer 26 to be communicatively coupled with other components.

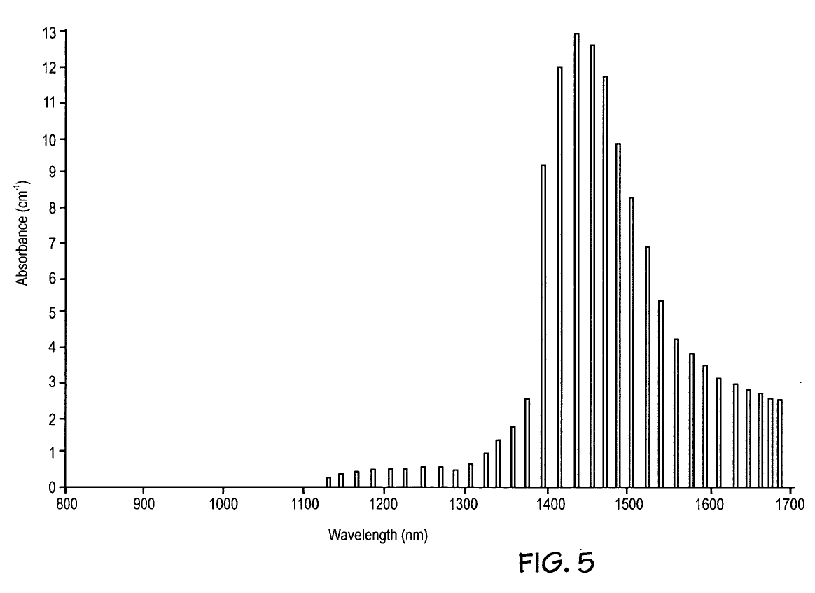

An exemplary spectrograph illustrating the spectral signature of water as detected by the solid state spectrometer 26 is shown in FIG. 5. As described above, the solid state spectrometer detects absorbance and reflectance of electromagnetic energy at narrow bands of discrete wavelengths, the combination of several or many of the bands may generate an absorbance spectrum. Specifically, a band may be a ten nanometer band of wavelengths, for example. As illustrated, water has a strong peak between 1400 and 1500 nm. As mentioned above, other analytes to be evaluated may absorb electromagnetic radiation near in other portions of the electromagnetic spectrum. The monitor 14 (FIG. 2) may be configured to determine the presence (or absence) of peaks by scanning the spectrum generated by the solid state spectrometer 26. The information gathered by analysis of the peaks may be used in the above mentioned algorithms or other algorithms, depending on the analyte of interest, to determine the relative water content of analyzed tissue.

The solid state spectrometer 26 is small and has no moving parts, providing reduced sensitivity to mechanical shock as compared to traditional spectroscopy instruments and micro-electro-mechanical systems (MEMS) discussed below. Additionally, the solid state detector array is low cost because of the wafer process used to make the detector. The low cost allows for the possibility of making the solid state detector array, and the entire sensor assembly disposable.

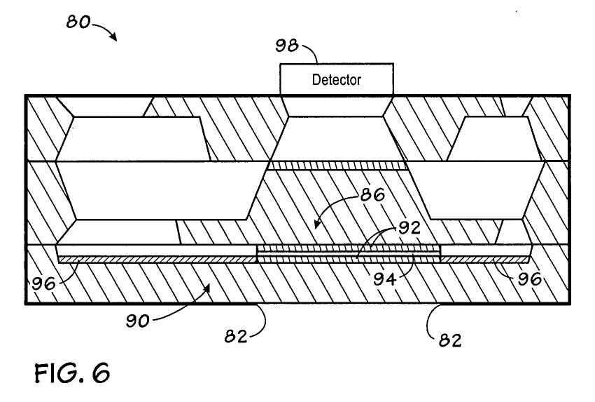

In an alternative exemplary embodiment, a micro-electro-mechanical systems (MEMS) detector may be implemented as the spectrum analyzer 24. Specifically, a MEMS detector may be implemented using micromirrors of a MEMS device having polymorphic layers. A cross-sectional view of a MEMS detector 80 is illustrated in FIG. 6 showing layers of silicon and/or silicon dioxide that form the structure of the MEMS device 80. The MEMS detector 80 includes an aperture 82 with an antireflective coating to allow electromagnetic radiation to enter the MEMS detector 80. The MEMS detector 80 has a reflector plate 86 suspended by a spring. The spring counteracts an electrostatic force caused by providing a voltage to driving electrodes 96. The voltage level is known and variable and is provided to driving electrodes 96 to control the size of an air cavity 94 between a reflector carrier 90 and the reflector plate 86.

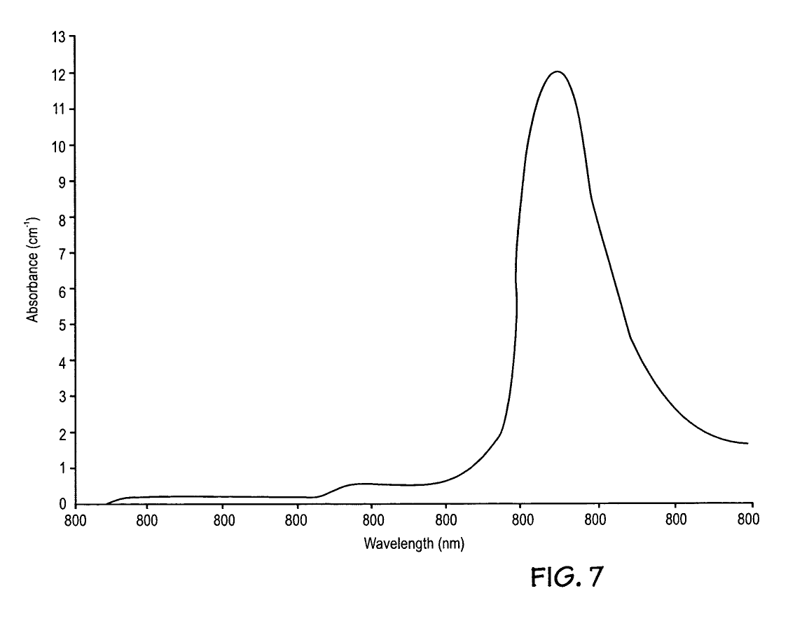

The size of the air cavity 94 determines the wavelength characteristics of light that are allowed to pass through the MEMS detector 80. Specifically, the frequency of light transmitted through the MEMS detector 80 generally has a known narrow distribution around a center wavelength or a center frequency. Changes in the size of the air cavity 94 changes the center frequency of the light that is transmitted through the MEMS detector 80. A photosensitive detector 98 may be used to determine the magnitude of the light that is transmitted through the MEMS detector 80. By adjusting the supplied voltage level, a signal of light intensity over or as a function of wavelengths or frequency can be generated. An exemplary spectrograph of the water signature generated by the MEMS detector 80 is illustrated in FIG. 7. As can be seen, the spectrograph is continuous and smooth throughout the range of detected wavelengths.

The monitor 14 has a microprocessor 28 which may be configured to calculate fluid parameters using algorithms known in the art or may be configured to compute the levels of other analytes, as mentioned above. The microprocessor 28 is connected to other component parts, such as a ROM 30, a RAM 32, and the control inputs 22. The ROM 30 may store the algorithms used to compute the physiological parameters. The RAM 32 may store values detected by the detector 18 for use in the algorithms.

Methods and algorithms for determining fluid parameters are disclosed in U.S. Pub. No. 2004-0230106, which has been incorporated herein by reference. Some fluid parameters that may be calculated include water-to-water and protein, water-to-protein, and water-to-fat. For example, in an exemplary embodiment the water fraction, f_{w}, may be estimated based on the measurement of reflectances, R(λ), at three wavelengths (λ₁=1190 nm, λ₂=1170 nm and λ₃=1274 nm) and the empirically chosen calibration constants c₀, c₁ and c₂ according to the equation:

f_{w}=c₂ log [R(λ₁)/R(λ₂)]+c₁ log [R(λ₂)/R(λ₃)]+c₀. (1)

In an alternative exemplary embodiment, the water fraction, f_{w}, may be estimated based on the measurement of reflectances, R(λ), at three wavelengths (λ=1710 nm, λ₂=1730 nm and λ₃=1740 nm) and the empirically chosen calibration constants c₀ and c₁ according to the equation:

$$fw = {C}_{1} \frac{log [ R ( {λ}_{1} ) / R ( {λ}_{2} ) ]}{Log [ R ( {λ}_{3} ) / R ( {λ}_{2} ) ]} + {C}_{0} . \qquad ( 2 )$$

Total tissue water accuracy better than ±0.5% can be achieved using Equation (2), with reflectances measured at the three closely spaced wavelengths. Additional numerical simulations indicate that accurate measurement of the lean tissue water content, f_{w}¹, can be accomplished using Equation (2) by combining reflectance measurements at 1125 nm, 1185 nm and 1250 nm.

In an alternative exemplary embodiment, the water content as a fraction of fat-free or lean tissue content, f_{w}¹, is measured. As discussed above, fat contains very little water so variations in the fractional fat content of the body lead directly to variations in the fractional water content of the body. When averaged across many patients, systemic variations in water content result from the variation in body fat content. In contrast, when fat is excluded from the calculation, the fractional water content in healthy subjects is consistent. Additionally, variations may be further reduced by eliminating the bone mass from the calculations. Therefore, particular embodiments may implement source detector separation (e.g. 1-5 mm), wavelengths of light, and algorithms that relate to a fat-free, bone-free water content.

In an alternative embodiment, the lean water fraction, f_{w}¹, may be determined by a linear combination of two wavelengths in the ranges of 1380-1390 nm and 1660-1680 nm:

f_{w}¹=c₂ log [R(λ₂)]+c₁ log [R(λ₁)]+c₀. (3)

Those skilled in the art will recognize that additional wavelengths may be incorporated into this or other calibration models in order to improve calibration accuracy.

In yet another embodiment, tissue water fraction, f_{w}, is estimated according to the following equation, based on the measurement of reflectances, R(λ), at a plurality of wavelengths:

$$fw = \frac{[ {\sum }_{n = 1}^{N} {p}_{n} log \{ R ( {λ}_{n} ) \} ] - [ {\sum }_{n = 1}^{N} {p}_{n} ] log \{ R ( {λ}_{N + 1} ) \}}{[ {\sum }_{m = 1}^{M} {q}_{m} log \{ R ( {λ}_{m} ) \} ] - [ {\sum }_{m = 1}^{M} {q}_{m} ] log \{ R ( {λ}_{M + 1} ) \}} , \qquad ( 4 )$$

where pₙ and qₘ are calibration coefficients. Equation (4) provides cancellation of scattering variances, especially when the N+1 wavelengths are chosen from within the same band (i.e. 950-1400 nm, 1500-1800 nm, or 2000-2300 nm).

Referring again to FIG. 2, as discussed above, keyboard 22 allows a user to interface with the monitor 14. For example, if a particular monitor 14 is configured to detect compartmental disorders as well as skin disorders, a user may input or select parameters, such as baseline fluid levels for the skin or a particular compartment of the body that is to be measured. Specifically, baseline parameters associated with various compartments or regions of the body or skin may be stored in the monitor 14 and selected by a user as a reference level for determining the presence of particular condition. Additionally, patient data may be entered, such as weight, age and medical history data, including, for example, whether a patient suffers from emphysema, psoriasis, etc. This information may be used to validate the baseline measurements or to assist in the understanding of anomalous readings. For example, the skin condition psoriasis would alter the baseline reading of skin water and, therefore, would affect any determination of possible bed sores or other skin wounds.

Detected signals are passed from the sensor 12 to the monitor 14 for processing. In the monitor 14, the signals are amplified and filtered by amplifier 33 and filter 36, respectively, before being converted to digital signals by an analog-to-digital converter 38. The signals may then be used to determine the fluid parameters and/or stored in RAM 32.

If a white light source is being used, a light drive unit 40 may not be used. However, if discrete wavelengths are implemented using LED emitters 24, the light drive unit controls the timing of the emitters 24. While the emitters 24 are manufactured to operate at one or more certain wavelengths, variances in the wavelengths actually emitted may occur which may result in inaccurate readings. To help avoid inaccurate readings, an encoder 42 and decoder 46 may be used to calibrate the monitor 20 to the actual wavelengths being used. The encoder 42 may be a resistor, for example, whose value corresponds to coefficients stored in the monitor 20. The coefficients may then be used in the algorithms. Alternatively, the encoder 42 may also be a memory device, such as an EPROM, that stores information, such as the coefficients themselves. Once the coefficients are determined by the monitor 14, they are inserted into the algorithms in order to calibrate the diagnostic system 10.

As mentioned above, the monitor 14 may be configured to display the calculated parameters on display 20. The display 20 may simply show the calculated fluid measurements for a particular region of tissue where the sensor has taken measurements. The fluid measurements may be represented as a ratio or a percentage of the water or other fluid present in the measured region.

It should be understood that the system 10 may be configured to take measurements from a single location on a patient's body and correlate the measurement to site specific hydration level, a whole body hydration index, or other values related to the hydration of an individual. Specifically, the system 10 may be placed along the centerline of the torso of a patient and a hydration index indicative of whole body hydration may be determined. In alternative applications, the system 10 may be configured to be placed on locations of a patient's body to test for localized conditions, such as compartmental edema or skin wounds, for example, as disclosed in U.S. Ser. No. 11/541,010, which is incorporated herein by reference.

A calibration technique may be implemented in conjunction with the sensor 12 and the transmission type sensor 40. The sensor 40 can be pre-calibrated during a manufacturing process. In the technique, the spectrum analyzer 24 is exposed to the electromagnetic radiation from the emitters 22 while a test object having a known spectral profile for the region of the electromagnetic spectrum that is of interest is placed in the light path. For example, Polytetrafluoroethylene (PTFE), commonly known as Teflon®, or a gold mirror may be used because each has known spectral properties for a broad range of the electromagnetic spectrum. The detected spectrum of the test object is compared against the standard or expected spectrum and the sensor is calibrated or zeroed so that the sensor 40 will reproduce the spectrum of test object. The calibration allows for the sensor to consistently repeat results of the probed tissue. The calibration may include determining or retrieving calibration factors or constants and providing them to the monitor 14 to calibrate to compensate for any instrument induced or other error.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention is to cover all modifications, equivalents, and alternatives falling within the spirit and scope of the invention as defined by the following appended claims.

What is claimed is:

1. A method for determining tissue hydration comprising:
transmitting electromagnetic radiation at tissue;
detecting the absorption spectrum of the tissue using a spectrum analyzer located in a sensor;
providing a signal correlative to the absorption spectrum from the spectrum analyzer to a monitor; and
processing the signal to determine an amount of water content in the tissue.

2. The method of claim 1 wherein transmitting electromagnetic radiation comprises transmitting a plurality of discrete wavelengths within the near-infrared (NIR) spectrum.

3. The method of claim 2, wherein transmitting the plurality of discrete wavelengths within the NIR spectrum comprises using three LEDs operating at different wavelengths between 1100 nm and 1400 nm.

4. The method of claim 1, wherein transmitting the electromagnetic radiation comprises using a broadband light source.

5. The method of claim 4, wherein the broadband light source emits white light.

6. The method of claim 1 wherein interpreting the spectrum comprises analyzing the distribution of spectral power to determine a ratio of water to other constituents.

7. The method of claim 1 comprising displaying the water content on a display.

8. The method of claim 7 wherein displaying the water content comprises displaying a ratio of water-to-other constituents as a percentage.

9. The method of claim 1 wherein the spectrum analyzer comprises a solid state spectrometer.

10. The method of claim 9 wherein the solid state spectrometer comprises filters to control the bandwidth of electromagnetic radiation that impinges on a detector array.

11. The method of claim 10 wherein the filters allow a 10 nm bandwidth of electromagnetic radiation impinge on the detector array.

12. The method of claim 1 wherein the spectrum analyzer comprises a micro-electro-mechanical system.

13. The method of claim 12 wherein the micro-electro-mechanical system comprises dielectric stack layers used to filter electromagnetic radiation.

14. A system for determining tissue constituents comprising:
a sensor comprising:
a source of electromagnetic radiation configured to transmit electromagnetic radiation at tissue;
a spectrum analyzer configured to detect the transmitted electromagnetic radiation and determine the spectral content of the detected electromagnetic radiation; and
a monitor communicatively coupled to the sensor and configured to receive and process the spectral content to determine the amount of water constituent present in the tissue.

15. The system of claim 14, wherein the spectrum analyzer comprising a solid state spectrum analyzer.

16. The system of claim 14 wherein the spectrum analyzer comprising a micro-electro-mechanical system (MEMS) device comprising a Fabry-Perot filter.

17. The system of claim 14 wherein the source of electromagnetic radiation is continuous spectrum light source.

18. The system of claim 14 wherein the continuous spectrum light source is a white light source.

19. The system of claim 14 wherein the source of electromagnetic radiation comprises a plurality of narrow band light sources.

20. The system of claim 19 wherein the plurality of narrow band light sources comprises light emitting diodes (LEDs) operating in the NIR band of the electromagnetic spectrum.
